(19) United States
(12) Patent Application Publication
(10) Pub. No.: US 2021/0015417 A1
(43) Pub. Date: Jan. 21, 2021

(54) EMOTION DATA TRAINING METHOD AND SYSTEM

(71) Applicant: Limbic Limited, London (GB)

(72) Inventors: Ross Edward Francis Harper, London (GB); Sebastiaan De Vries, London (GB)

(21) Appl. No.: 16/982,997

(22) PCT Filed: Mar. 21, 2019
(86) PCT No.: PCT/GB2019/050816

§ 371 Date: Sep. 21, 2020

(30) Foreign Application Priority Data
Mar. 21, 2018 (GB) 1804537.7
Jan. 28, 2019 (GB) 1901158.4

(51) Int. Cl. A61B 5/16; A61B 5/00; G06N 20/00

(52) U.S. Cl. CPC A61B 5/165; G06N 20/00; A61B 5/6802; A61B 5/486

(57) ABSTRACT

The present invention relates to a computer implemented method for training one or more parameters of a model. More particularly, the present invention relates to a computer implemented method for training one or more parameters of a model based on emotion signals. Aspects and/or embodiments seek to provide a computer implemented method which can calculate and/or predict emotion signals for training software implementations of mathematical models or machine learned models based on these emotion signals.

### FIELD

The present invention relates to a computer implemented method for training one or more parameters of a model. More particularly, the present invention relates to a computer implemented method for training one or more parameters of a model based on emotion signals.

### BACKGROUND

Emotion detection is a new field of research, blending psychology and technology, and there are currently efforts to develop, for example, facial expression detection tools, sentiment analysis technology and speech analysis technology in this field of research.

If emotion detection can be made to work robustly, applications can include social robots, autonomous cars and emotion based digital interactions. However, the subconscious and natural way that emotion is expressed, which provides a non-verbal, unbiased and unfiltered way to assess how humans interact with what surrounds them, as well as how they interact with technology, is very complex and difficult to assess using present methods.

In order to dive deeper into the human connection with technology, especially in order to develop more efficient and effective ways of assisting humans, there is currently a need to determine the emotions of users robustly and then determine how their technology can best use this information.

### SUMMARY OF INVENTION

Aspects and/or embodiments seek to provide a computer implemented method which can calculate and/or predict emotion signals for training software implementations of mathematical models or machine learned models based on these emotion signals.

According to a first aspect, there is provided a computer implemented method for training one or more parameters of a main model, wherein the main model comprises an objective function, the method comprising the steps of: predicting or calculating one or more emotion signals using an emotion detection model; inputting said one or more emotion signals into said main model; inputting one or more training data into said main model; optimising an objective function of the main model based on the one or more emotional signals and the one or more training data; and determining the one or more parameters based on the optimised objective function of the main model.

Providing trained learnt models, which are trained using emotion signals from an already trained emotion detection model, which can be used by developers in any platform to integrate emotion-based optimisation into their systems or applications, can allow for the use of emotion data with technology in a variety of applications.

Optionally, further comprising a step of regularisation based on the one or more emotion signals.

Optionally, the step of regularisation comprises adapting the objective function: optionally wherein the objective function comprises a loss function.

The step of regularisation based on the one or more emotion signals can generalise the function to fit data from other sources or other users.

Optionally, further comprising inputting any or any combination of: one or more physiological data; text data and/or video data. Optionally, the one or more emotion signals are determined from one or more physiological data. Optionally, the one or more physiological data is obtained from one or more sources and/or sensors. Optionally, the one or more sensors comprise any one or more of: wearable sensors; audio sensors; and/or image sensors. Optionally, the one or more physiological data comprises one or more biometric data. Optionally, the one or more biometric data comprise any one or more of: skin conductance; skin temperature; actigraphy; body posture; EEG; and/or heartbeat data from ECG or PPG.

A variety of input data as physiological data can be used.

Optionally, one or more data related to the one or more emotion signals over time is extracted from the one or more physiological data.

Optionally, the main model comprises one or more machine learning models. Optionally, the one or more data related to the one or more emotion signals over time is input into the one or more machine learning models.

Optionally, the one or more machine learning models comprises any one or more of: regression models; regularised models; classification models; probabilistic models, deep learning models; and/or instance-based models.

Optionally, the one or more emotion signals comprise one or more of: classification and/or category-based emotion signals; overall emotion signals; emotional response; predicted emotional response; continuous emotion signals; and/or end emotion signals.

The one or more emotion signals comprising one or more of: classification-based emotion signals; overall emotion signals; emotional response; predicted emotional response; continuous emotion signals; and/or end emotion signals, can be used to further optimise the training of the main model(s).

Optionally, the main model optimises an outcome of one or more tasks: optionally wherein the one or more tasks is unrelated to detection of emotion. Optionally, the one or more physiological data is stored as training data for the emotion detection model and/or the one or more emotion signals is stored as training data for the main model.

The training data and/or the output of the trained emotion detection model may be used for the learning of other machine learning classifiers seeking to optimise a task using emotion signals.

According to a second aspect, there is provided one or more learnt models output from the method for training the one or more parameters of the main model.

According to a third aspect, there is provided a use of the one or more learnt models.

According to a fourth aspect, there is provided an apparatus operable to perform the method of any preceding feature.

According to a fifth aspect, there is provided a system operable to perform the method of any preceding feature.

According to a sixth aspect, there is provided a computer program operable to perform the method and/or apparatus and/or system of any preceding feature.

### BRIEF DESCRIPTION OF DRAWINGS

Embodiments will now be described, by way of example only and with reference to the accompanying drawing having like-reference numerals, in which:

FIG. 1 shows an overview of the training process for one or more parameters of a model;

FIG. 2 illustrates a typical smart watch;

FIG. 3 illustrates the working of an optical heart rate sensor on the example typical smart watch of FIG. 2;

FIG. 4 illustrates a table of sample emotion-eliciting videos that can be used during the training process for the model of the specific embodiment;

FIG. 5 illustrates the structure of the model according to the specific embodiment;

FIG. 6 illustrated the probabilistic classification framework according to the model of the embodiment shown in FIG. 5; and

FIG. 7 illustrates the coupling of an emotion detection/prediction model with a main model.

### SPECIFIC DESCRIPTION

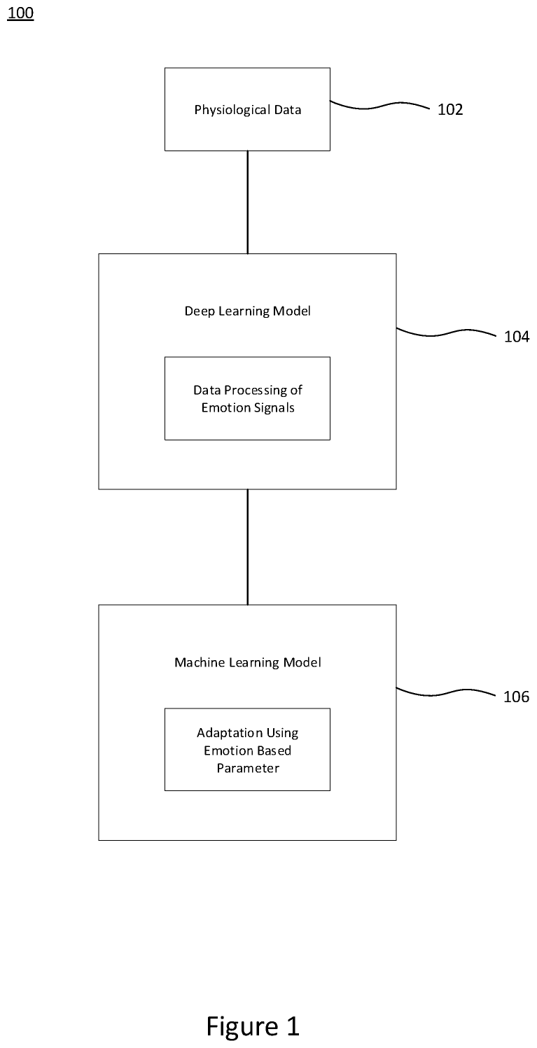

Referring to FIG. 1, example embodiments for a computer implemented method for training one or more parameters of a (main) model using emotion signals will now be described. The term main model is used here to distinguish it from the emotion detection model, but can also simply be read as model.

In this embodiment, physiological data as shown as 102 may consist multiple varieties of data collected from detection systems. Physiological data may include, but is not limited to the scope of, image data, audio data and/or biometric data. Examples of such data include, skin conductance, skin temperature, actigraphy, body posture, EEG, heartbeat, muscle tension, skin colour, noise detection, data obtained using eye tracking technology, galvanic skin response, body posture, facial expression, body movement, and speech analysis data obtained through speech processing techniques.

In some embodiments the term physiological data is intended to refer to autonomic physiological data: i.e. peripheral physiological signals of the kind that can be collected by a wearable device. Examples of this type of data include ECG, PPG, EEG, GSR, temperature, and/or breathing rate among others. In these embodiments or in other embodiments, it is also intended that the term physiological data is intended to refer to behavioural physiological data: for example, behavioural signals such as facial expression, voice, typing speed, text/verbal communication and/or body posture among others.

In an embodiment, emotion signals may be extracted from physiological data received or collected using a camera, wearable device or a microphone etc. for example by means of a mobile device, personal digital assistant, a computer, personal computer or laptop, handheld device or a tablet, a wearable computing device such as a smart watch. All of which may be capable of detecting a physiological characteristic of a particular user of the device.

In an embodiment, physiological data obtained over a period of time for a user is input into a machine learning emotion detection model such as, but not limited to, deep learning models, reinforcement learning models and representation learning models. For example, a deep learning model such as a long short-term memory recurrent neural network (LSTM RNN), as shown as 104, may be implemented. The implemented deep learning model may be learnt to process the input physiological data such as extracting temporal data from the physiological data. In an example of a user providing their heartbeat, RR values or inter-beat intervals (IBIs) may be extracted from the obtained heartbeat signal via a sensor over a course of time. The IBI values are used to predict emotion signals which can represent the emotional state or emotional states of the user. In other examples, an emotional time series i.e. the emotion signal may be extracted from a physiological time series i.e. the signal generated from the received data via image, audio or wearable devices/sensors. In such examples, emotion signals can be extracted as appropriate to the type of data received in order to classify and/or predict the emotional state or emotional states of a user. Physiological data collected may be processed within different time frames for the emotion experienced by the user of the physiological data.

In this embodiment, biometric information (or physiological information or data, which can be either autonomic or behavioural) can be collected from a wearable device strapped to a user, or extracted from video footage by measuring minute changes in facial flushing, or via other methods or a combination of methods. By having obtained a biometric time series, an emotion-based time series can be constructed with an emotion detection model i.e. the deep learning model such as the LSTM.

In an embodiment, a training signal which is optimised for AI or machine learning corresponds to emotion signals.

In an embodiment, emotional time series i.e. the emotion signals, are used to train classifier algorithms. The classifier algorithm used e.g. Logistic Regression, may be further modified by means of a regularisation of the (main) model which adds an emotion-based parameter for the optimisation of a learning model to regularise it based on emotion signals. The algorithm using a regularised (main) model may seek to learn a parameter which minimises unwanted characteristics. For example, in a situation where happiness of a user is sought for optimisation, the sadness of the user may be minimised through algorithm modification of a loss function. Using regularised algorithms which conventionally penalise models such as the logistic regression model based on parameter complexity may help to generalise a model for new datasets i.e. the adaptation to a loss function within any suitable model by means of using an emotion signal-based parameter which can be generalised.

Regression algorithms which may be used include but are not limited to, Ordinary Least Squares Regression (OLSR), Linear Regression, Logistic Regression, Stepwise Regression, Multivariate Adaptive Regression Splines (MARS) and Locally Estimated Scatterplot Smoothing (LOESS). The output of emotion signals forms all or part of the input to train such regression algorithms.

In an embodiment, the emotion parameter which is added to the algorithm may take into account emotion signals of a user within various time frames. User emotion may be added as a sum over individual emotional state moments on a per classification basis, or by measuring the overall accumulated emotional state of the user, or the user's emotional state solely at the end of training.

In an embodiment, a variety of other algorithms which focus on the addition of a emotion based parameter may be implemented. Such algorithms may include for example Instance-based algorithms which compare new data points against an existing database according to a similarity-based measure. Examples of instance-based algorithms include, k-Nearest Neighbour (k-NN), Learning Vector Quantisation (LVQ), Self-Organising Map (SOM) and Locally Weighted Learning (LWL).

In an embodiment, learnt (main) models may be used by developers in any platform in order to incorporate learned approaches into their digital products. Developers may implement a set of instructions such as computer based code into an application and use signals obtained via a cloud through an Application Programming Interface (API) or via a user interface through a Software Development Kit (SDK) be it either directly on the hardware for through a software package which may be installed on the device. In other embodiments, implementation may be via a combination of both API and SDK.

In an embodiment, processed emotion signals from deep learning algorithms may be used as input to train other classifiers wherein the output emotion data may be used for training other machine learning models whether in the cloud or offline. In such cases, signals may not need to be obtained via an API or an SDK.

Many applications are possible for use with emotion training data and approaches as set out above. For example, with (a) emotion data that can be used to train machine learning (main) models and other learnt models and (b) an approach that allows for training of machine learning (main) models and other learnt (main) models to use emotion data; example applications of training and trained (main) models can include: predicting medicines or therapeutic interventions recommended/needed/that might be effective for a user based on their emotion data; use with computer games and the emotion data of a game-player; advertising, in particular the response to adverts by a viewer or target user; driverless cars, where a driverless car can learn to drive in a style that suits the passenger for example by slowing down to allow the passengers to view a point of interest, or driving slower than necessary for a passenger that is nervous; and any smart device seeking to learn behaviours that optimise a positive mental state in the human user (e.g. virtual assistants).

In an embodiment dealing with a straightforward example, an example dealing with how emotional signals can be incorporated and used in relation to a computer game will now be described.

This example involves an autonomous agent within a computer game having the purpose of getting from A to B as fast as possible. The autonomous agent within the computer game can collect rewards in the form of gold coins. However, the autonomous agent within the computer game can also fall into a hole, ending the journey/game. Naturally, one would want the autonomous agent to find the fastest way to get from A to B, but also maximising the number of gold coins collected while minimising the chances of falling into a hole.

One way to solve this problem would be through reinforcement learning, for example using the Q-learning algorithm. Q-learning is a model-free reinforcement learning algorithm. The goal of Q-learning is to learn a policy, which tells the agent what action to take under the circumstances (i.e. state). The agent needs to learn to maximise cumulative future reward (henceforth “R”). In the following notation, we shall use a subscript t to denote the rewards from a particular time step. A reward equation would thus be expressed as:

$${R}_{t} = {r}_{t + 1} + {r}_{t + 2} + {r}_{t + 3} + … = {\sum }_{k = 0}^{∞} {r}_{t + k + 1} \qquad ( 2 )$$

To avoid the reward function going to infinity, future reward is discounted. Conceptually, this accounts from future rewards being less certain than more immediate rewards. Therefore, this can be expressed in the following equation:

$${R}_{t} = {r}_{t + 1} + γ {r}_{t + 2} + {γ}^{2} {r}_{t + 3} + … = {\sum }_{k = 0}^{∞} {γ}^{k} {r}_{t + k + 1} \qquad ( 3 )$$

where 0<γ<1.

A policy, written π(s, a), describes a way of acting. It is a function that takes in a state and an action, and returns the probability of taking that action in that state. Therefore, for a given state, it must be true that Σₐ π(s, a)=1.

Our goal in reinforcement learning is to learn an optimal policy, π*. An optimal policy is a policy which tells us how to act to maximise return in every state.

To learn the optimal policy, value functions are used. There are two types of value functions that are used in reinforcement learning: the state value function, denoted V(s), and the action value function, denoted Q(s,a).

The state function describes the value of a state when following a policy. It is the expected reward when starting from state s acting according to our policy it:

V^{π}(s)= [symbol] _{π}[Rₜ|sₜ=s] (4)

The other value function we will use is the action value function. The action value function tells us the value of taking an action in some state when following a certain policy. It is the expected return given the state and action under it:

Q^{π}(s,a)= [symbol] _{π}[Rₜ|sₜ=s,aₜ=a] (5)

Using Bellman equations and dynamic programming, one can learn the parameters of the above equations that optimise discounted future reward equation (3).

In the above example, the reward is calculated using an intrinsic understanding of the problem: that collecting gold coins is desired whereas falling into a hole is not desired. An embodiment with a modified example will now be presented where the purpose in this example of getting from A to B, collecting gold coins and avoiding holes is replaced with a purpose that is measured by the emotional state of another agent. An example is where the autonomous agent is a hotel concierge and the other agent is a human guest.

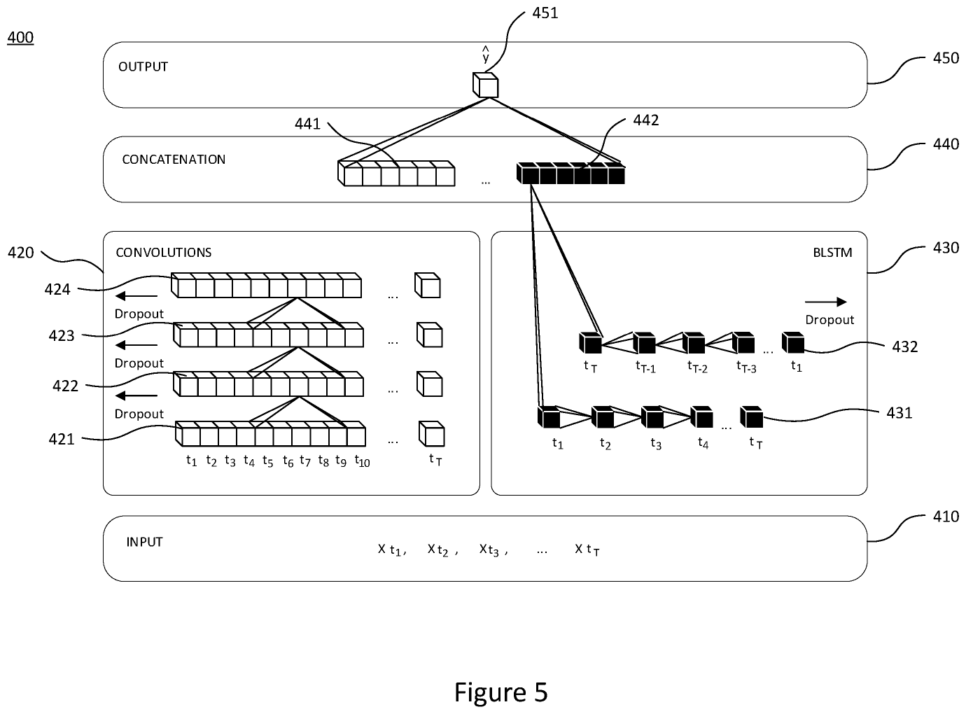

Referring to FIG. 5, which will be described in further detail below, an end-to-end emotion detection model architecture 400 is shown where data flows through two temporal processing streams: one-dimensional convolutions 420 and a bi-directional LSTM 430. The output from both streams is then concatenated 441, 442 before passing through a dense layer to output a regression estimate for valence.

Using existing approaches, it is not necessarily clear how to optimise the modified example set out above relating to an autonomous hotel concierge agent, where the agent is seeking to maximise the emotional state of a human guest. One might speculate that the concierge should take actions to be attentive for example by asking how it might be of service, or remaining nearby the guest should its assistance be requested/needed. However, what works for one guest might not work for another, so it is unclear which behaviours should be optimised in this situation.

It is proposed to introduce a new reward signal: human emotion. To obtain this signal, we construct a separate mathematical emotion detection model designed to infer emotion from physiological signals (either autonomic or behavioural) signals. This model can take the form of that according to other aspects and/or embodiments for example the embodiment described in connection with FIG. 5.

In an embodiment using reinforcement learning, the reward at each time step, rₜ, is simply the output of the emotion detection model at each time step, ŷₜ:

$${R}_{t} = {\hat{Y}}_{t} = {\hat{y}}_{t + 1} + {\hat{y}}_{t + 2} + {\hat{y}}_{t + 3} + … = {\sum }_{k = 0}^{∞} {\hat{y}}_{t + k + 1} \qquad ( 6 )$$

The time step of inputs to the emotion detection model need not be the same as the time steps for the reinforcement learning problem (for example, the emotion detection model may require input every millisecond, whereas the reinforcement learning model may operate at the minute time scale).

In this and other embodiments, the reward signal in the reinforcement learning paradigm is equal to, or is replaced with, the output of a separate emotion detection model. This can couple the goal of the autonomous reinforcement learning agent with the emotional state of a human, allowing the autonomous agent to optimise for the emotional state of the human, rather than some alternative defined goal based on insight into the task at hand as per the previous example.

V^{π}(s)= [symbol] _{π}[Ŷₜ|sₜ=s] (7)

Q^{π}(s,a)= [symbol] _{π}[Ŷₜ|sₜ=s,aₜ=a] (8)

It should be noted that the described embodiment relates to the use of reinforcement learning algorithms, however the same principle can be applied to other machine learning paradigms and other learned models and applications, for example in any or any combination of: logistic regression; regression models; regularisation models; classification models; deep learning models; instance-based models; Ordinary Least Squares Regression (OLSR), Linear Regression, Logistic Regression, Stepwise Regression, Multivariate Adaptive Regression Splines (MARS) and Locally Estimated Scatterplot Smoothing (LOESS).

Therefore, in other embodiments using different machine learning models with emotion prediction being used as a training signal, a common loss function might take the form:

$${θ}^{*} = {argmin}_{θ} \frac{1}{N} {\sum }_{i = 1}^{N} {( {z}_{i} - {\hat{z}}_{i} )}^{2} + λ \cdot Φ ( θ ) = {argmin}_{θ} \frac{1}{N} {\sum }_{i = 1}^{N} {( {z}_{i} - f ( {x}_{i} , θ ) )}^{2} + λ \cdot Φ ( θ ) \qquad ( 9 )$$

where zᵢ is the output of the model (e.g. a neural network or logistical regression), θ* are the learned model parameters, θ are the parameters of the model to be learned, ŷᵢ=f(xᵢ, θ) is the model output given the current parameters, Φ(θ) is a regularisation (or penalty) term, and λ is the regularisation term coefficient.

The regularisation term is included to stop the model parameters growing too large (and thus over-fitting the data). However, it is possible to construct a new regularisation function Φ that is a function of both the model parameters and the output of the emotion detection model Φ(θ, yₜ):

$${θ}^{*} = {argmin}_{θ} \frac{1}{N} {\sum }_{i = 1}^{N} {( {z}_{i} - f ( x , θ ) )}^{2} + λ \cdot Φ ( θ , {\hat{y}}_{t} ) \qquad ( 10 )$$

In this case, the learned model parameters would be influenced by the emotional state of a human from which ŷₜ was generated.

In a further embodiment, providing measures of mental wellness and/or emotion using a wearable device will be described, using the sensors now typically provided on smart watches and fitness bands, and would provide the ability to monitor both individual users as well as populations and groups within populations of users. This provides a substantially continuous non-invasive emotion detection system for one or a plurality of users.

For example, heart rate variability (HRV) is a biomarker that is straightforward to calculate using existing sensors on wearable devices and can be used to quantify physiological stress. As described above, it is possible to use sensors such as optical heartrate sensors to determine a wearer's heartbeat time series using a wearable device. More specifically, because activity in the sympathetic nervous system acts to trigger physiological changes in a wearer of a device associated with a “fight or flight” response, the wearer's heartbeat becomes more regular when this happens, thus their HRV decreases. In contrast, activity in the antagonistic parasympathetic nervous system acts to increase HRV and a wearer's heartbeat becomes less regular. Thus, it is straightforward to determine HRV using a wearable device by monitoring and tracking a wearer's heartbeat over time. It is however currently difficult to determine whether the changes in HRV that can be detected are mentally “positive”, i.e. indicate eustress, or mentally “negative”, i.e. indicate distress, as HRV may change in the same way for a variety of positive or negative reasons—therefore monitoring solely HRV does not provide a meaningful determination of a wearer's mental state.

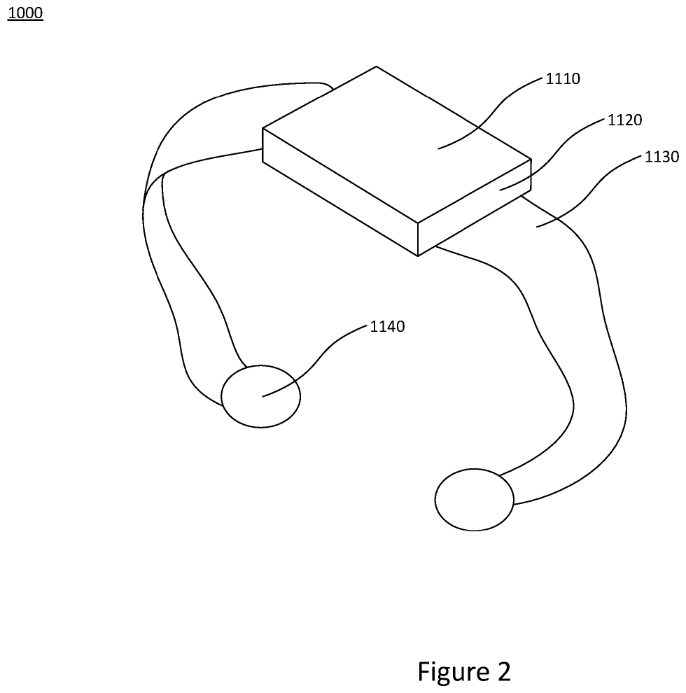

Referring to FIG. 2, a typical smartwatch 1100 that can be used to provide emotion signals/emotion data for training (main) models is shown and will now be described. The smartwatch 1100 is provided with an optical heart rate sensor (not shown) integrated into the body 1120, a display 1110 that is usually a touchscreen to both display information and graphics to the wearer as well as allow control and input by a user of the device, and a strap 1140 to attach the device to a wearer's wrist.

In alternative embodiments, other wearable devices in place of a smartwatch 1100 can be used, including but not limited to fitness trackers and rings.

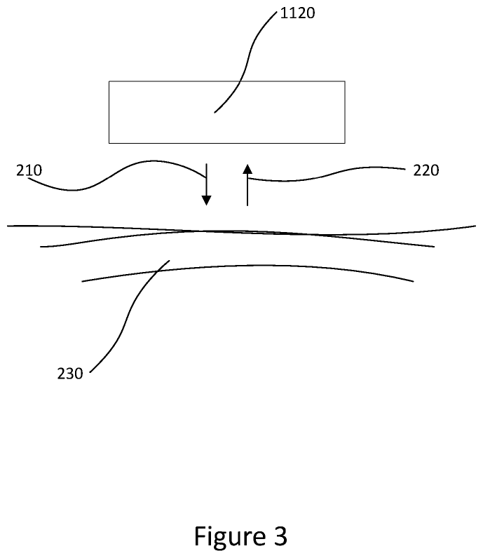

Referring to FIG. 3, the optical emitter integrated into the smartwatch body 1120 of FIG. 2 emits light 210 into the wearer's arm 230 and then any returned light 220 is input into the optical light sensor integrated in the smartwatch body 1120.

Further sensors, as outlined above, can be integrated into the smartwatch body 1120 in alternative embodiments.

In the present embodiment, a deep learning neural network emotion detection model is trained on users with smartwatches 1100. The input data to the emotion detection model from the smartwatches 1100 is the inter-beat intervals (IBI) extracted from the photoplethysmography (PPG) time series.

In other embodiments, other input data can be used instead, or in combination with the IBI from the PPG time series. For example, but not limited to, any or any combination of: electrodermal activity data; electrocardiogram data; respiration data and skin temperature data can be used in combination with or instead of the IBI from the PPG time series. Alternatively, other data from the PPG time series can be used in combination with or instead of the IBI from the PPG time series or the other mentioned data.

The emotion detection model uses a deep learning architecture to provide an end-to-end computation of the emotional state of a wearer of the smartwatch 1100 directly based on this input data. Once the emotion detection model is trained, a trained emotion detection model is produced that can be deployed on smartwatches 1100 that works without needing further training and without needing to communicate with remote servers to update the emotion detection model or perform off-device computation.

Referring to FIG. 5, the example deep learning neural network emotion detection model 400 is structured as follows according to this embodiment:

The example deep learning neural network model provides an end-to-end deep learning model for classifying emotional valence from (unimodal) heartbeat data. Recurrent and convolutional architectures are used to model temporal structure in the input signal.

Further, there is provided a procedure for tuning the model output depending on the threshold for acceptable certainty in the outputs from the model. In applications of affective computing (i.e. automated emotion detection), this will be important in order to provide predictive interpretability for the model, for example in domains such as healthcare (where high certainty will be required, and so it is better not to output a classification with low certainty) or other domains (where a classification is needed, even if it only has a low certainty).

The example deep learning neural network model is structured in a sequence of layers: an input layer 410; a convolution layer 420; a Bidirectional Long Short-Term Memory Networks (BLSTM) layer 430; a concatenation layer 440; and an output layer 450.

The input layer 410 takes the information input into the network and causes it to flow to the next layers in the network, the convolution layer 420 and the BLSTM layer 430.

The convolution layer 420 consist of multiple hidden layers 421, 422, 423, 424 (more than four layers may be present but these are not be shown in the Figure), the hidden layers typically consisting of one or any combination of convolutional layers, activation function layers, pooling layer, fully connected layers and normalisation layers.

A Bayesian framework is used to model uncertainty in emotional state predictions. Traditional neural networks can lack probabilistic interpretability, but this is an important issue in some domains such as healthcare. In an embodiment, neural networks are re-cast as Bayesian models to capture probability in the output, In this formalism, network weights belong to some prior distribution with parameters a Posterior distributions are then conditioned on the data according to Bayes' rule:

$$p ( θ \vert D ) = \frac{p ( D \vert θ ) p ( θ )}{p ( D )} \qquad ( Equation 1 )$$

where D is the data.

While useful from a theoretical perspective, Equation 1 is infeasible to compute. Instead, the posterior distributions can be approximated using a Monte-Carlo dropout method (alternatively embodiments can use methods including Monte Carlo or Laplace approximation methods, or stochastic gradient Langevin diffusion, or expectation propagation or variational methods). Dropout is a process by which individual nodes within the network are randomly removed during training according to a specified probability. By implementing dropout at test and performing N stochastic forward passes through the network, a posterior distribution can be approximated over model predictions (approaching the true distribution as N→∞). In the embodiment, the Monte-Carlo dropout technique is implemented as an efficient way to describe uncertainty over emotional state predictions.

The BLSTM layer 430 is a form of generative deep learning where two hidden layers 431, 432 of opposite directions are connected to the same output to get information from past (the “backwards” direction layer) and future (the “forwards” direction layer) states simultaneously. The layer 430 functions to increase the amount of input information available to the network 400, and provide the functionality of providing context for the input layer 410 information (i.e. data/inputs before and after, temporally, the current data/input being processed).

The concatenation layer 440 concatenates the output from the convolution layer 420 and the BLSTM layer 430.

The output layer 450 then outputs the final result 451 for the input 410, dependent on whether the output layer 450 is designed for regression or classification. If the output layer 450 is designed for regression, the final result 451 is a regression output of continuous emotional valence and/or arousal. If the output layer 450 is designed for classification, the final result 451 is a classification output, i.e. a discrete emotional state.

Data flows through two concurrent streams in the emotion detection model 400. One stream comprises four stacked convolutional layers that extract local patterns along the length of the time series. Each convolutional layer is followed by dropout and a rectified linear unit activation function (i.e. converting the output to a 0 or a 1). A global average pooling layer is then applied to reduce the number of parameters in the model and decrease over-fitting. The second stream comprises a bi-directional LSTM followed by dropout. This models both past and future sequence structure in the input. The output of both streams are then concatenated before passing through a dense layer to output a regression estimate for valence.

In order to capture uncertainty in the model predictions, dropout is applied at test time. For a single input sample, stochastic forward propagation is run N times to generate a distribution of model outputs. This empirical distribution approximates the posterior probability over valence given the input time series. At this point, a regression output can be generated by the model.

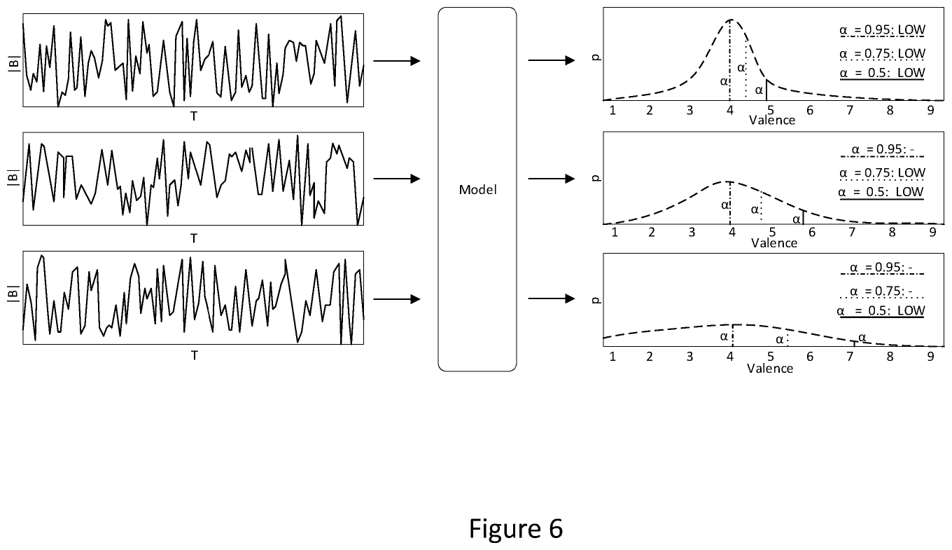

To generate a classification output, i.e. to translate from a regression to a classification scheme, decision boundaries in continuous space need to be introduced. For a binary class problem, this decision boundary is along the central point of the valence scale to delimit two class zones (high and low valence for example). Next a confidence threshold parameter a is used to tune predictions to a specified level of model uncertainty. For example, when α=0.95, at least 95% of the output distribution must lie in a given class zone in order for the input sample to be classified as belonging to that class (see FIG. 6). If this is not the case, then no prediction is made. The model may therefore not classify all instances, so the model only outputs a classification when the threshold that has been predetermined is met. As a increases, the model behaviour moves from risky to cautious but with less likelihood that a classification will be output (but with more certainty for classifications that are output). For binary classifications, there will always be at least 50% of the output distribution that will be within one of the two prediction zones, thus when α=0.5 the classification is determined by the median of the output distribution and a classification will always be made.

In other embodiments, variations of this network structure are possible but require the deep neural network model to model time dependency such that it uses the previous state of the network along, and/or temporal information within the input signal, to output a valence score. Other neural network structures can be used.

The training process for the emotion detection model in the embodiment works as follows:

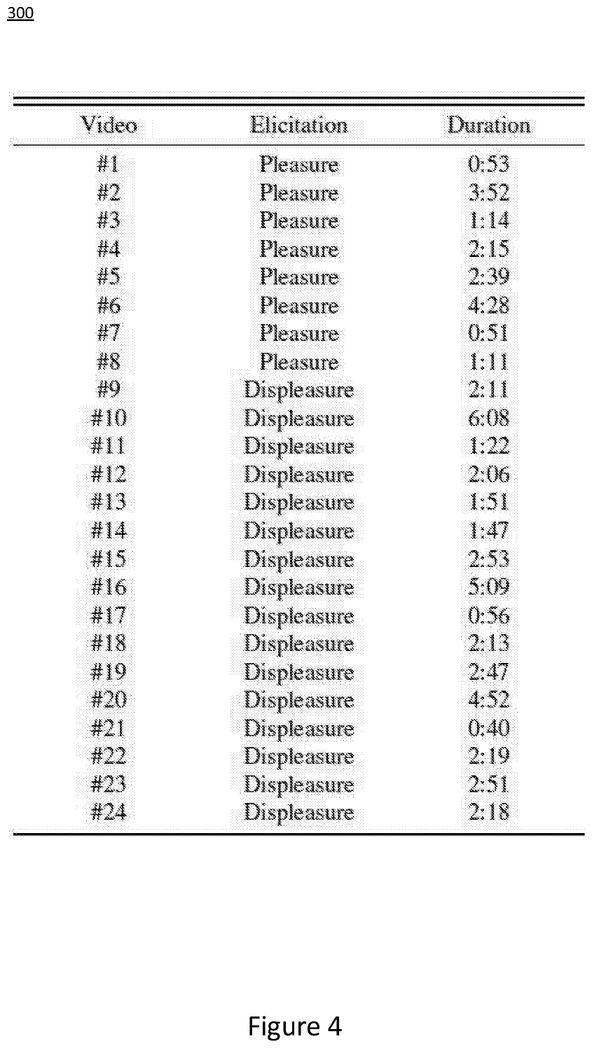

Referring to FIG. 4, users wearing a wearable device such as the smartwatch 1100 are exposed to emotion-eliciting stimuli (e.g. video stimuli) that has been scored independently for its ability to induce both pleasurable and displeasurable feelings in viewers. The table 300 in FIG. 4 shows a table of 24 example video stimuli along with an associated pleasure/displeasure rating for each video and a length of each video.

In the embodiment where the stimuli are video stimuli, each user watches the series of videos and, after each video, each user is asked to rate their own emotional state for pleasure and displeasure in line with the “valence” metric from the psychological frameworks for measuring emotion (e.g. the popular self-assessment Manikin (SAM) framework). A statistically significant sample size of users will be needed. Additionally, a one-minute neutral video following each user completing their rating of their emotional state should allow the user to return to a neutral emotional state between viewing the next emotion-eliciting video. Further, playing the video sequence in a different random order to each user should improve the training process.

It will be understood that other options for stimuli are possible to carry out this process. In some embodiments, other options for training are possible in order to collect input-output pairs, where the input data is a physiological data time series and the output data (to which the input data is paired) is user emotional state (this data can be self-reported/explicit or inferred from analysing users using text and/or facial data and/or speech or other user data).

Referring to FIG. 5, and once the emotion detection model has been training, a standalone output model is produced that can be deployed on a wearable device to predict the emotional state of a user of the wearable device on which the model is deployed.

Additionally, the model is able to predict the emotional state of a user even where the specific input data hasn't been seen in the training process. The predicted emotional state is output with a confidence level by the model. Bayesian neural network architectures can be used in some embodiments to model uncertainty in the model parameters and the model predictions. In other embodiments, probabilistic models capable of describing uncertainty in their output can be used.

As described above, other types of learned algorithm can be used apart from that described in the embodiments.

In some embodiments, the learned algorithm can also output confidence data for the determined emotional state of the user of the wearable device, as sometimes it will be highly probable that a user is in a particular emotional state given a set of inputs but in other situations the set of inputs will perhaps only give rise to a borderline determination of an emotional state, in which case the output of the algorithm will be the determined emotional state but with a probability reflecting the level of uncertainty that this is the correct determined emotional state.

All suitable types of format of wearable device are intended to be usable in embodiments, provided that the wearable device has sufficient hardware and software capabilities to perform the computation required and be configured to operate the software to perform the embodiments and/or alternatives described herein. For example, in some embodiments the wearable device could be any of a smartwatch; a wearable sensor; a fitness band; smart ring; headset; smart textile; or wearable patch. Other wearable device formats will also be appropriate, as it will be apparent.

In some embodiments, should the wearable device have location determination capabilities, for example using satellite positioning or triangulation based on cell-towers or Wi-Fi access points, then the location of the wearable device can be associated with the user's emotional state can be determined.

In some embodiments, some of the processing to use the emotion detection model can be done remotely and/or the model/learned algorithm can be updated remotely and the model on the wearable device can be updated with the version that has been improved and which is stored remotely. Typically, some form of software updating process run locally on the wearable device will poll a remote computer which will indicate that a newer model is available and allow the wearable device to download the updated model and replace the locally-stored model with the newly downloaded updated model. In some embodiments, data from the wearable device will be shared with one or more remote servers to enable the model(s) to be updated based on one or a plurality of user data collected by wearable devices.

In some embodiments, the emotional states being determined include any or any combination of discrete emotions such as: depression; happiness; pleasure; displeasure; and/or dimensional emotions such as arousal and valence.

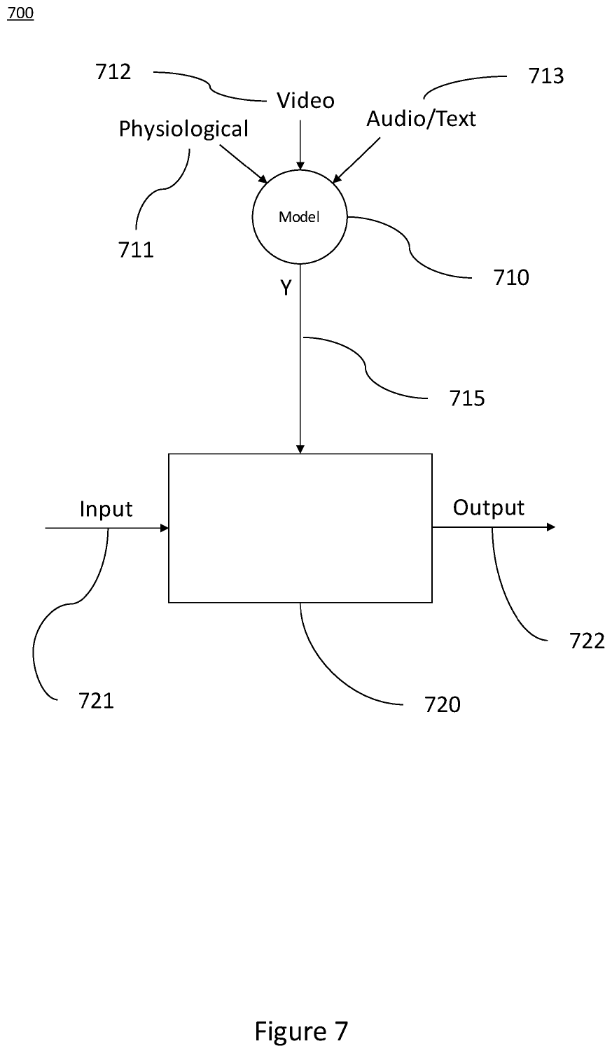

Referring now to FIG. 7, the combination of emotion detection model 710 and main model 720 as described in the above embodiments is shown.

Here, the input data 711, 712, 713 (which may include any or any combination of autonomic physiological data 711, video data 712, audio data and/or text data 713) is provided to the emotion detection model 710. The emotion detection model 710 outputs Y, the emotion detected and/or predicted 715, from the input data 711, 712, 713 into the main model 720 as a parameter or input to the main model 720. Main model 720 then uses this detected and/or predicted emotion data 715 when operating on the input data 721 input to the main model 720 in order to produce output data 722.

The emotion detection model 710 can take one of a variety of possible forms, as described in the above embodiments, suffice that it outputs an emotional state prediction or detection for use with the main model 720.

Any system features as described herein may also be provided as method features, and vice versa. As used herein, means plus function features may be expressed alternatively in terms of their corresponding structure.

Any feature in one aspect may be applied to other aspects, in any appropriate combination. In particular, method aspects may be applied to system aspects, and vice versa. Furthermore, any, some and/or all features in one aspect can be applied to any, some and/or all features in any other aspect, in any appropriate combination.

It should also be appreciated that particular combinations of the various features described and defined in any aspects can be implemented and/or supplied and/or used independently.

What is claimed is:

1. A computer implemented method for training one or more parameters of a main model, wherein the main model comprises an objective function, the method comprising the steps of:
predicting or calculating one or more emotion signals using an emotion detection model;
inputting said one or more emotion signals into said main model;
inputting one or more training data into said main model;
optimising an objective function of the main model based on the one or more emotional signals and the one or more training data; and
determining the one or more parameters based on the optimised objective function of the main model.

2. The method of claim 1 further comprising a step of regularisation based on the one or more emotion signals.

3. The method of claim 2 wherein the step of regularisation comprises adapting the objective function: optionally wherein the objective function comprises a loss function.

4. The method of claim 1 further comprising inputting any or any combination of: one or more physiological data; text data and/or video data.

5. The method of claim 1 wherein the one or more emotion signals are determined from one or more physiological data.

6. The method according to claim 4 wherein the one or more physiological data is obtained from one or more sources and/or sensors.

7. The method of claim 6 wherein the one or more sensors comprise any one or more of: wearable sensors; audio sensors; and/or image sensors.

8. The method of claim 7, wherein the one or more physiological data comprises one or more biometric data.

9. The method of claim 8 wherein the one or more biometric data comprise any one or more of: skin conductance; skin temperature; actigraphy; body posture; EEG; and/or heartbeat data from ECG or PPG.

10. The method of claim 1 wherein one or more data related to the one or more emotion signals over time is extracted from the one or more physiological data.

11. The method of claim 1 wherein the main model comprises one or more machine learning models.

12. The method of claim 11 wherein the one or more data related to the one or more emotion signals over time is input into the one or more machine learning models.

13. The method of claim 11 wherein the one or more machine learning models comprises any one or more of: regression models; regularised models; classification models; probabilistic models; deep learning models; and/or instance-based models.

14. The method of claim 1 wherein the one or more emotion signals comprise one or more of: classification and/or category-based emotion signals; overall emotion signals; emotional response; predicted emotional response; continuous emotion signals; and/or end emotion signals.

15. The method of claim 1 wherein the main model optimises an outcome of one or more tasks: optionally wherein the one or more tasks is unrelated to detection of emotion.

16. The method of claim 1 wherein the one or more physiological data is stored as training data for the emotion detection model and/or the one or more emotion signals is stored as training data for the main model.

17. One or more learnt models output from the method of claim 1.

18. Use of the one or more learnt models of claim 17.

19. A computer program product operable to perform the method of claim 1.
